(19) United States
(12) Patent Application Publication
(10) Pub. No.: US 2007/0052926 A1
(43) Pub. Date: Mar. 8, 2007

(54) Eye drop aimer system

(76) Inventor: Yusheng Li, Tianjin (CN)

Correspondence Address: Yusheng Li, 4-8-501, Guangshun Yuan, Guangdong Road, Hexi Dist, Tianjin, 300204, CN

(21) Appl. No.: 11/465,782

(22) Filed: Aug. 18, 2006

Related U.S. Application Data
(60) Provisional application No. 60/714,781, filed on Sep. 7, 2005.

(51) Int. Cl. A61B 3/10

(52) U.S. Cl. 351/212

(57) ABSTRACT

The eye drop aimer system is used in connection with an eye drop container. The eye drop aimer system utilizes an aim instrument having at least one reflective surface. In a preferred embodiment, the reflective surface of the aim instrument is located vertically around the eye drop spout of the eye drop container, when the user visually aims with his or her reflected eye image in the reflective surface and the eye drop spout end, his or her head and eye are in a substantially horizontal position and the eye drop container is substantially vertically disposed, the position of the spout end seen in the reflected eye image in the reflective surface indicates the aim of the drops in the eye; a light source instrument having at least one light source is utilized to supply lighting when the users drop eye in dark and weak.

This is a non-provisional patent application of U.S. provisional patent application APPL No. 60/714,781, filed on Sep. 7, 2005.

The present invention relates to a mirrored eye drop aimer system and a method of directing eye drops into the eye of a user.

### BACKGROUND OF THE INVENTION

Eye drops are widely used, but many users simply guess, often soaking the eyelid, lashes, and cheek but missing the eye completely. Some users simply lack the aim necessary, some users must ask for help from others. The inconveniences are unsanitary. Eye drop devices which comprise mirrors are known. U.S. Pat. No. 3,913,575 to Windsor discloses an eye dropper device which includes a swing out mirror attached to the base of the eye dropper. U.S. Pat. No. 6,325,781 B1 to Muroff discloses a mirrored eye dropper target. The mirrored end of the target is in a singular plane entirely tangential with respect to the eye dropper body. These systems can not satisfy that the users visually aim directly in the direction of the axial centerline of the eye dropper spout from the spout end, and can not supply lighting. The present invention overcomes these difficulties.

### OBJECTS OF THE INVENTION

It is an object of the present invention to provide a mirrored eye drop aimer system and a method for directing eye drops to an eye of a user.

It is an additional object of the present invention to provide an eye drop aimer system which utilizes an aim instrument having at least one reflective surface, the reflective surface is located around the periphery of the eye drop spout of the eye drop container.

It is an additional object of the present invention to provide an eye drop aimer system wherein the user visually aims with his or her reflected eye image in the reflective surface and the eye drop spout end such that when his or her head and eye are in a substantially horizontal position and the eye drop container is substantially vertically disposed, the position of the eye drop spout end seen in the reflected eye image in the reflective surface indicates the aim of the drops in the eye.

It is an additional object of the present invention to provide an eye drop aimer system which utilizes a light source instrument comprising at least one light source; the light source instrument is connected with the eye drop container and/or the aim instrument. The light source supply lighting when the users drop eye in dark and weak.

### BRIEF SUMMARY OF THE INVENTION

The eye drop aimer system is used in connection with an eye drop container. The eye drop aimer system utilizes an aim instrument having at least one reflective surface. In a preferred embodiment, the reflective surface of the aim instrument is located vertically around the eye drop spout of the eye drop container, when the user visually aims with his or her reflected eye image in the reflective surface and the eye drop spout end, his or her head and eye are in a substantially horizontal position and the eye drop container is substantially vertically disposed, the position of the spout end seen in the reflected eye image in the reflective surface indicates the aim of the drops in the eye; a light source instrument having at least one light source is utilized to supply lighting when the users drop eye in dark and weak.

### BRIEF DESCRIPTION OF THE SEVERAL VIEWS OF THE DRAWING

Further objects and advantages of the present invention can be found in the detailed description of the preferred embodiments when taken in conjunction with the accompanying drawings in which:

FIG. 1 is a sectional view of a preferred embodiment of the present invention taken along the axial centerline 0 of the eye drop spout 7.

FIG. 2 is a sectional view of another preferred embodiment of the present invention taken along the axial centerline 0 of the eye drop spout 7.

FIG. 3 is a sectional view of another preferred embodiment of the present invention taken along the axial centerline 0 of the eye drop spout 7.

FIG. 4 is a sectional view of another preferred embodiment of the present invention taken along the axial centerline 0 of the eye drop spout 7.

### DETAILED DESCRIPTION OF THE PREFERRED EMBODIMENTS

The present invention relates to a mirrored eye drop aimer system and a method of directing eye drops into the eye of a user.

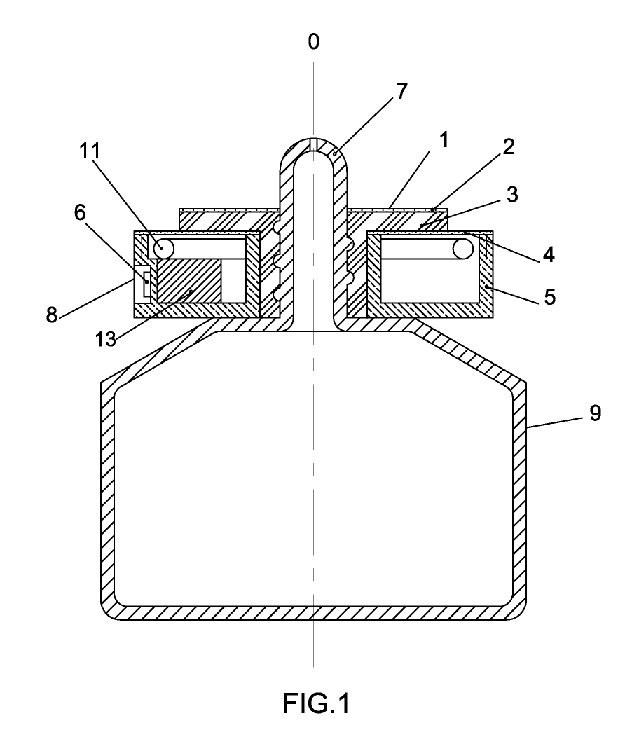

FIG. 1 diagrammatically illustrates an eye drop aimer system mounted onto an eye drop container 9. In the preferred embodiment, the major components of the eye drop aimer system include an aim instrument 1 and a light source instrument 8. The aim instrument 1 comprises a reflective surface 2 and a means 3 having an axial center hole ringing around eye drop spout 7 of the eye drop container 9. The means 3 locates the reflective surface 2 vertically around the periphery of the eye drop spout 7, additionally close to the end of the eye drop spout 7. The eye drop spout 7 has an axial center line 0.

In the preferred embodiment, the light source instrument 8 is located between the aim instrument 1 and the eye drop container 9. Other location of the light source instrument 8 on the eye drop container 9 and/or on the aim instrument 1 may be utilized. The major components of the light source instrument 8 include a body 5 having an axial center hole ringing around the periphery of the mean 3 of the aim instrument 1, a light source 11, a switch 6 and an energy source 13. The switch 6 links the light source 11 with the energy source 13. More light sources may be utilized. The light source 1 the switch 6 and the energy source 13 are located in/on the body 5. Surface 4 of the body 5 faces towards the end of the eye drop spout 7. The surface 4 is at least partly transparent. The outside diameter of the surface 4 is bigger than the reflective surface 2. In operation, the light from the light source 11 comes around the reflective surface 2 through the transparent part of the surface 4. The preferred shape of the reflective surface 2 and surface 4 is round with a center hole. Other shapes may be utilized. The aim instrument 1 may be the part that features the light source instrument 8 on the eye drop container 9. The aim instrument 1 and light source instrument 8 could be installed on the eye drop container 9 together or separately.

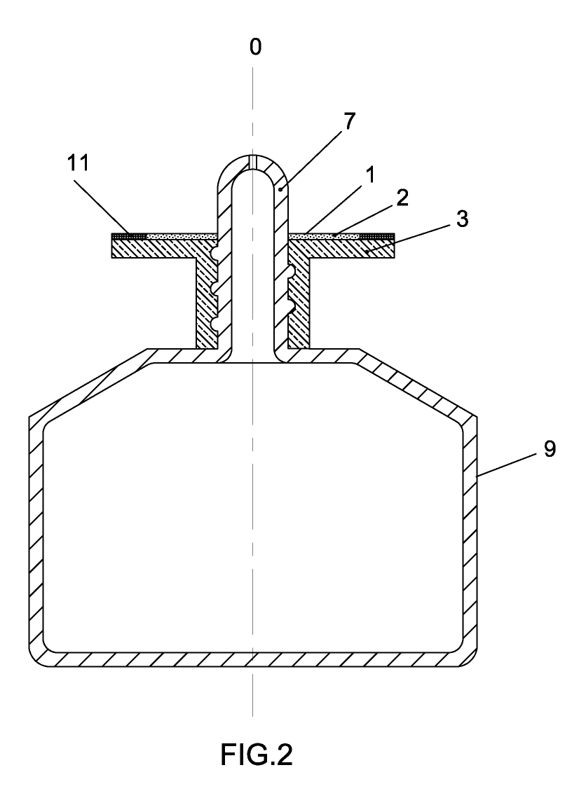

FIG. 2 diagrammatically illustrates another embodiment of the present invention. In FIG. 2, the major components of the eye drop aimer system include an aim instrument 1 and a self illuminant light source 11 located on it. In the preferred embodiment, the aim instrument 1 comprises a reflective surface 2 and a means 3 having an axial center hole ringing around the eye drop spout 7 of the eye drop container 9. The means 3 locates the reflective surface 2 vertically around the periphery of the eye drop spout 7; additionally close to the end of the eye drop spout 7. The eye drop spout 7 has an axial center line 0. The location of the self illuminant light source 11 on the aim instrument 1 may be around the reflective surface 2. Other location may be utilized. Additionally more self illuminant light source may be utilized. The means 3 may be eliminated and the reflective surface 2 and/or self illuminant light source 11 may be located directly on the eye drop container 9. The self illuminant light source 11 may be tritium (3H), fluorescence and other types of self illuminant substances.

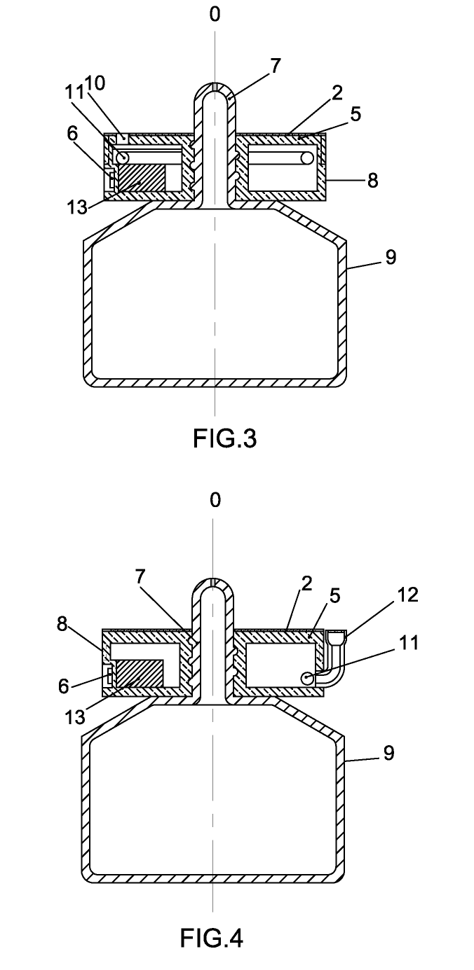

FIGS. 3 and 4 diagrammatically illustrate other embodiments of the present invention. In FIG. 3, the major components of the eye drop aimer system include a light source instrument 8 and a reflective surface 2 located on it. In the preferred embodiment, the light source instrument 8 includes a light source 11, a switch 6 and an energy source 13 in/on light source instrument body 5. The switch 6 links the light source 11 with the energy source 13. The light source instrument body 5 has an axial center hole ringing around the eye drop spout 7 and locates the reflective surface 2 vertically around the periphery of the eye drop spout 7, additionally close to the end of the eye drop spout 7. The eye drop spout 7 has an axial center line 0. A hole 10 is located on the reflective surface 2 and/or the surface of the light source instrument body 5 toward the end of eye drop spout 7. In operation, the light from the light source 11 comes through the hole 10. The hole 10 may be only located on the light source instrument body 5, additionally beside the reflective surface 2. More holes may be utilized.

In FIG. 4 the major components of the eye drop aimer system include a light source instrument 8 and a reflective surface 2 located on it. In the preferred embodiment, the light source instrument 8 includes a light source 11, a switch 6 and an energy source 13 in/on light source instrument body 5 and a tube 12 is located on the side well of the light source instrument body 5. The switch 6 links the light source 11 with the energy source 13. The light source instrument body 5 has an axial center hole ringing around the eye drop spout 7 and locates the reflective surface 2 vertically around the periphery of the eye drop spout 7, additionally close to the end of the eye drop spout 7. The eye drop spout 7 has an axial center line 0. The light from the light source 11 come through the tube 12. Alternatively, the tube 12 can be eliminated and the light source 11 may be located on the side well of the light source instrument body 5 and/or the surface of the light source instrument body 5 toward the end of eye drop spout 7 and/or on the reflective surface 2. Other location of the light source 11 on the light source instrument 8 and/or on the aim instrument 1 and/or on the eye drop container 9 may be utilized.

In the preferred embodiments shown in FIGS. 1,2,3,4, the light source 11 may be lamp, LED, tritium (3H), fluorescence or other type of light substance. The light should be perceptible and not harmful to eyes. The energy source 13 for the light source 11 except the self illuminant light source 11 shown in FIG. 2 may be battery. Other type of energy source may be utilized. The reflective surface 2 may be mirror, reflective film, reflective plating or other type of reflective substance or layer. Additionally, the reflective surface 2 may be a reflective singular surface or components of several reflective surfaces in a singular plane. Preferably, the means 3 of the aim instrument 1 or body 5 of light source instrument 8 connects the eye drop spout 7 with the structure on the eye drop container 9 that link the cover of eye drop spout end to the eye drop container 9, the structure may be screw or reed or clip. Other way of connecting may be utilized.

When the user visually aims with his or her reflected eye image in the reflective surface 2 and the end of eye drop spout 7, his or her head and eye are in a substantially horizontal position and the eye drop container 9 is substantially vertically disposed, the fluid falls into the eye upon ejection from the spout end. The position of the end of eye drop spout 7 seen in the reflected eye image in the reflective surface 2 indicates the aim of the drops in the eye. By adjusting the position between the end of the eye drop spout 7 and the reflection of the eye, the user could accurately place the drops in the eye. The light source 11 supplies lighting when the users drop eye in dark and weak. Both the reflective surface 2 of the aim instrument 1 and the light source 11 of the light source instrument 8 are installed on the eye drop container 9, only one hand is needed for use. To fit the various shapes of eye drop containers, the installation of the aim instrument 1 and/or the light source instrument 8 on the eye drop container 9 could be removable or immovable.

While the invention has been described with reference to certain forms and embodiments thereof, it is apparent that many other forms and embodiments will be obvious to those skilled in the art in view of the foregoing description. Thus, the scope of the invention should not be limited by that description and the proportion of the figures but, rather, only by the claims appended hereto.

I claim:

1. A method of directing eye drops to an eye of a user comprising the steps of:
Providing an eye drop container with an eye drop spout with a spout end, said eye drop container holding fluid to be delivered into said eye of said user via said spout and spout end;
Establishing an aim instrument having at least a reflective surface located around the periphery of said eye drop spout of said eye drop container such that when said user visually aim with his or her reflected eye image in said reflective surface and the end of said eye drop spout, his or her head and eye are in a substantially horizontal position and said eye drop container is substantially vertically disposed, the position of said eye drop spout end seen in the reflected eye image in said reflective surface indicates the aim of the drops in said eye.

2. A method as claimed in claim 1 including the step of establishing a light source instrument comprising at least one light source and connecting said light source instrument with said eye drop container and/or with said aim instrument.

3. A method as claimed in claim 1 wherein said eye drop spout has an axial center line and said reflective surface is disposed entirely in a vertical plane with respect to said eye drop spout.

4. A method as claimed in claim 2 wherein said aim instrument and/or said light source instrument is installed on said eye drop container either together or separately.

5. A method as claimed in claim 2 wherein the installation of said aim instrument and/or said light source instrument on said eye drop container is either removable or immovable.

6. A method as claimed in claim 2 wherein said eye drop spout has an axial center line and said reflective surface is disposed entirely in a vertical plane with respect to said eye drop spout.

7. A method as claimed in claim 2 wherein said aim instrument and/or said light source instrument connects said eye drop spout with a structure on the eye drop container that link the cover of said eye drop spout end to said eye drop container.

8. A method as claimed in claim 6 wherein said reflective surface is located close to the end of said eye drop spout.

9. A method as claimed in claim 6 wherein said aim instrument is said reflective surface locating on said light source instrument.

10. A method as claimed in claim 6 wherein said light source instrument is at least one self illuminant light source locating on said aim instrument.

11. A method as claimed in claim 6 wherein said reflective surface and/or at least one self illuminant light source is located directly on said eye drop container without other parts for connecting.

12. A method as claimed in claim 6 wherein the shape of said reflective surface and the surface of said light source instrument toward said eye drop spout end is round with a center hole.

13. A method as claimed in claim 6 wherein said light source instrument is located between said reflective surface and said eye drop container, said light source instrument defines at least a light source, an energy source, a switch and a body.

14. A method as claimed in claim 13 wherein said light source, said energy source and said switch are located in/on said body of said light source instrument, said light source instrument has an axial center hole ringing around the periphery of a means of said aim instrument, said means is for connecting with said eye drop container, the surface of said light source instrument toward said eye drop spout end is at least partly transparent, the outside diameter of said surface of said light source instrument is bigger than said reflective surface.

15. A method as claimed in claim 13 wherein said light source, said energy source and said switch are located in/on said body of said light source instrument, said light source instrument has an axial center hole ringing around the periphery of said eye drop spout, said reflective surface and/or the surface of said light source instrument body toward the end of said eye drop spout defines at least a hole where light from said light source come through.

16. A method as claimed in claim 13 wherein said light source, said energy source and said switch are located in/on said body of said light source instrument, a tube is located on side well of said light source instrument body, said light source instrument has an axial center hole ringing around the periphery of said eye drop spout, light from said light source come through said tube.

17. A method as claimed in claim 13 wherein said energy source and said switch are located in/on the body of said light source instrument, said light source is located on the said reflective surface.
